(19) United States
(12) Patent Application Publication
(10) Pub. No.: US 2025/0223515 A1
(43) Pub. Date: Jul. 10, 2025

(54) HARD SURFACE CLEANING COMPOSITIONS COMPRISING RHAMNOLIPIDS AND SARCOSINATE CO-SURFACTANT

(71) Applicant: Stepan Company, Northbrook, IL (US)

(72) Inventors: Ronald A. Masters, Wheeling, IL (US); Vanessa Demarco, Skokie, IL (US)

(21) Appl. No.: 19/089,749

(22) Filed: Mar. 25, 2025

Related U.S. Application Data
(63) Continuation of application No. PCT/US23/74996, filed on Sep. 25, 2023.
(60) Provisional application No. 63/410,064, filed on Sep. 26, 2022.

(51) Int. Cl. C11D 1/74; C11D 1/10; C11D 1/37; C11D 3/22; C11D 17/04

(52) U.S. Cl. CPC C11D 1/74; C11D 1/10; C11D 1/37; C11D 3/22; C11D 17/049; C11D 2111/14; C11D 2111/42

(57) ABSTRACT

Hard surface cleaning compositions that contain at least one rhamnolipid, at least one alkyl sarcosinate anionic surfactant, and at least one builder are disclosed. The compositions provide a combination of good soil removal and low film/streak properties, and are useful for soil removal applications including, but not limited to, cleaning bathroom and kitchen articles and/or surfaces.

### BACKGROUND OF THE INVENTION

The present technology, in general, relates to hard surface cleaning compositions that comprise biolipids, such as rhamnolipids. More particularly, the present technology relates to hard surface cleaning compositions comprising rhamnolipids and an alkyl sarcosinate anionic co-surfactant that provide both good cleaning performance and low film/streak properties.

Desirable attributes for hard surface cleaning compositions, in general, include the ability to emulsify or penetrate greasy or oily soils and suspend or disperse particulates, in order to clean articles or surfaces; and then prevent the soils, grease, or particulates from re-depositing on the newly cleaned surfaces. It is also desirable for the hard surface cleaning compositions to have low streaking or filming on the cleaned surface. However, cleaning compositions that provide good surface cleaning often result in poor film/streak properties. Reducing the amount of surfactants in the cleaning compositions can lessen the amount of filming/streaking, but often at the expense of cleaning performance. It has been a challenge, for example, for the detergent industry to provide a product that provides effective cleaning performance while minimizing film formation and streaking. Often, compositions providing good cleaning properties result in poor film/streak properties, and vice-versa.

There has also been a recent trend to formulate products with ingredients that are based on renewable raw materials rather than fossil fuels. Such ingredients are considered “green” or “natural”, since they are derived from renewable and/or sustainable sources. As a result, they are more environmentally friendly than ingredients derived from fossil fuels. An ingredient having a high Biorenewable Carbon Index (BCI), such as greater than 80, indicates that the ingredient contains carbons that are derived primarily from plant, animal or marine-based sources.

Rhamnolipids are interface-active glycolipids produced by various bacterial species, and are an example of a “green” ingredient, since they can be prepared by means of fermentation based on renewable raw materials. The use of glycolipids, including rhamnolipids, in detergent and other cleaning compositions is known in the art. For example, EP3663386 discloses that glycolipid surfactants in combination with another surfactant can provide good cleaning performance. The reference mentions a wide variety of cationic, anionic, and amphoteric surfactants that can be combined with the glycolipid surfactants, and discloses wide ranges for the amounts of glycolipids and additional surfactant that can be used. The reference does not discuss compositions that have low film/streak properties. Another reference, WO2019/034490, discloses the use of a particular rhamnolipid mixture to improve the washing power of detergents and cleaning products. However, the reference does not mention film/streak properties or describe compositions that provide good cleaning and low filming/streaking.

It would be desirable to have hard surface cleaner compositions that include actives derived from renewable sources, such as rhamnolipids, but that can also deliver good cleaning performance while providing low filming/streaking. Providing a hard surface cleaning composition comprising rhamnolipids would satisfy sustainability goals of ensuring sustainable consumption through the use of bio-based surfactant materials. Surprisingly, the present technology demonstrates one or more formulations of hard surface cleaning compositions comprising rhamnolipids that provide good cleaning ability while providing low film/streak formation.

### BRIEF SUMMARY OF THE INVENTION

In one aspect, the present technology provides a hard surface cleaning composition comprising at least one rhamnolipid in an amount of 0.10% to 0.55% active weight, based on the total weight of the composition; at least one alkyl sarcosinate anionic surfactant in an amount of 0.25% to less than 0.7% active weight, based on the total weight of the composition, at least one builder or chelant in an amount of 0.1% to less than 0.50% active weight based on the total weight of the composition; and one or more carriers in an amount to total 100% by weight of the composition, wherein the at least one builder comprises gluconic acid, sodium gluconate, or a combination thereof.

In another aspect, the present technology provides a hard surface cleaning composition comprising from 0.10% to 0.55% active weight, based on the total weight of the composition, of at least one rhamnolipid; from 0.25% to less than 0.7% active weight, based on the total weight of the composition, of at least one alkyl sarcosinate anionic surfactant; from 0.1% to less than 0.50% active weight, based on the total weight of the composition, of at least one builder or chelant comprising gluconic acid, sodium gluconate, or a combination thereof; and one or more carriers in an amount to total 100% by weight of the composition, wherein the hard surface cleaning composition provides at least 75% cleaning by Stroke 7, measured in accordance with ASTM D4488-95 A5, and film/streak on ceramic of 1.0 or less and on polypropylene of 3.0 or less, measured in accordance with the method for determining film/streak disclosed in US Published Application No. 2021/0139815, where the results reported here are divided by a factor of 10.

In some embodiments, the hard surface cleaning composition can be in the form of a solid or liquid concentrate that is diluted prior to use at dilution ratios of 1:64, 1:32, 1:16, or 1:10, among others, before it can be applied to articles, substrates, or surfaces to be cleaned. When the hard surface cleaner composition is in the form of a dilutable concentrate or powder, the composition comprises at least one rhamnolipid in an amount of about 1% to about 36% by weight, based on the weight of the composition, at least one alkyl sarcosinate surfactant in an amount of about 2.5% to less than 45% by weight, based on the weight of the composition, and at least one builder in an amount of 1% to less than 32% by weight based on the weight of the composition.

Yet a still further aspect of the present technology provides a method of cleaning a hard surface comprising the steps of contacting at least one soiled surface with a cleaning composition comprising from 0.10% to 0.55% active weight, based on the total weight of the composition, of at least one rhamnolipid; from 0.25% to less than 0.7% active weight, based on the total weight of the composition, of at least one alkyl sarcosinate anionic surfactant; from 0.1% to less than 0.50% active weight, based on the total weight of the composition, of at least one builder or chelant comprising gluconic acid, sodium gluconate, or a combination thereof; and one or more carriers in an amount to total 100% by weight of the composition; and removing the composition and soil from the surface.

### BRIEF DESCRIPTION OF THE DRAWINGS

FIG. 1 is a 3-D surface plot prepared from the hard surface cleaning formulations in Table 1.

FIG. 2 is an alternative view of the 3-D surface plot prepared from the hard surface cleaning formulations in Table 1.

FIG. 3 is a contour plot prepared from the hard surface cleaning formulations in Table 1.

### DETAILED DESCRIPTION OF THE INVENTION

### Definitions

“Biorenewable Carbon Index” (BCI) refers to a calculation of the percent carbon derived from a biorenewable resource and is calculated based on the number of biorenewable carbons divided by the total number of carbons in the entire molecule.

“Biorenewable” is defined herein as originating from animal, plant, or marine material.

The terms “active”, “% active”, and “% active weight” refer to the amount of the active ingredient without regard to the amount of water or other solvent that may be present with the ingredient.

A “ready-to-use” or “RTU” product, composition or formulation of the present technology refers to a product, composition, or formulation that is ready to be applied to articles or surfaces to be cleaned.

A “dilutable,” “concentrate,” or “dilutable concentrate” product, composition, or formulation of the present technology refers to a product, composition, or formulation that needs to be diluted with a diluent (e.g., water) in a ratio of, for example, 1:64, 1:32, 1:16, or 1:10, among others, before it can be applied to articles, substrates, or surfaces to be cleaned.

As defined herein, a “rhamnolipid” is a glycolipid that has a lipid portion that includes one or more, typically linear, saturated or unsaturated β-hydroxy-carboxylic acid moieties and a saccharide portion of one or more units of rhamnose.

The saccharide portion and the lipid portion are linked via a β-glycosidic bond between the 1-OH group of a rhamnose moiety of the saccharide portion and the 3-OH group of a β-hydroxy-carboxylic acid of the lipid portion. Thus, the carboxylic acid of one carboxylic acid moiety defines the end of the rhamnolipid. Where more than one rhamnose-moiety is included in a rhamnolipid, each of the rhamnose moieties not linked to the lipid portion is linked to another rhamnose moiety via a 1,4β-glycosidic bond. In embodiments where two or more β-hydroxy-carboxylic acids are present in a rhamnolipid, the β-hydroxy-carboxylic acid moieties are selected independently from each other. β-hydroxy carboxylic acid moieties may in some embodiments be identical. In some embodiments, they are different from each other.

“Film” or “filming” as used herein refers to a residue that remains after cleaning and covers a surface.

“Streak” or “streaking” as used herein refers to a residue that remains after cleaning and forms lines or spots on the surface.

“Low filming/streaking” as used herein refers to a composition that provides a film/streak score of 1.0 or less when tested on a ceramic surface according to the method for determining film/streak disclosed in US Published Application No. 2021/0139815, but with the results divided by a factor of 10.

A composition that provides “good cleaning” or “effective cleaning” for the present technology is one that removes at least 75% of soil by Stroke 7 as determined in accordance with ASTM D4488-95 A5. The NGO certification for Green Seal GS-37 Standard for Cleaning Products for Industrial and Institutional Use states that General Purpose Cleaners shall remove at least 80% of the particulate soil in ASTM D4488-95, A5. The compositions that reach 75% by stroke 7 achieve 80% cleaning by stroke 10.

The present technology generally relates to hard surface cleaning compositions that comprise at least one rhamnolipid, at least one alkyl sarcosinate anionic surfactant, and at least one builder that provides the desired grease and soil removal from a surface, as well as low filming/streaking of the surface. Not to be bound by any particular theory, it is believed that the specific combination of these specific components in specific amounts provides the desired combination of effective cleaning and low streak/film formation. If the amounts of the components are not within the particular disclosed ranges, the resulting composition may not provide the desired combination of properties. Similarly, any of the components alone, or a composition that uses a different surfactant component, may not provide the combination of properties. The specific ratios of components required for obtaining the combination of good cleaning and low film/streak can be different with different combinations of rhamnolipid and co-surfactants, and are not obvious or predictable. The ranges for combined good film/streak and cleaning performance tend to be relatively narrow and thus only discernible after substantial research effort.

The hard surface cleaning compositions of the present technology comprise at least one rhamnolipid. The rhamnolipid may have the following structure (I):

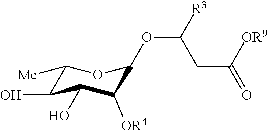

In this formula, R⁹ is a hydrogen atom (H) or an aliphatic group that has a main chain of one to about 46, such as one to about 42, one to about 40, one to about 38, one to about 36, one to about 34, one to about 30, one to about 28, including e.g. 2, 3, 4, 5, 6, 7, 8, 9, 10, 11, 12, 13, 14, 15, 16, 17, 18, 19, 20, 21, 22, 23, 24, 25, 26, 27 or 28 carbon atoms and one to about three, including two, oxygen atoms. In some embodiments, the main chain of the respective aliphatic group carries a terminal carboxylic acid group and/or an internal ester group. As an illustrative example in this regard, R⁹ may be of the formula-CH(R⁵)—CH2-COOR⁶. In these illustrative moieties, R⁵ may be an aliphatic moiety with a main chain that has a length from 1 to about 19, such as from 1 to about 17, from 1 to about 15, from 1 to about 13, about 2 to about 13, about 3 to about 13 or about 4 to about 13, including e.g. 2, 3, 4, 5, 6, 7, 8, 9, 10, 11 or 12 carbon atoms. R⁴ in formula (I) is a hydrogen atom (H), or a rhamnopyranosyl moiety. Re is a hydrogen atom.

The term “aliphatic” means, unless otherwise stated, a straight or branched hydrocarbon chain, which may be saturated or mono- or poly-unsaturated and include heteroatoms. The term “heteroatom” as used herein means an atom of any element other than carbon or hydrogen. Herein, an unsaturated aliphatic group contains one or more double bonds (alkenyl moieties). The branches of the hydrocarbon chain may include linear chains as well as non-aromatic cyclic elements. The hydrocarbon chain may, unless otherwise stated, be of any length, and contain any number of branches. Typically, the hydrocarbon (main) chain includes 1 to about 5, to about 10, to about 15 or to about 20 carbon atoms. Examples of alkenyl moieties are straight-chain or branched hydrocarbon moieties that contain one or more double bonds. Alkenyl moieties generally contain about two to about twenty carbon atoms and one or more, for instance two, double bonds, such as about two to about ten carbon atoms, and one double bond. Examples of alkyl groups are methyl, ethyl, propyl, butyl, pentyl, hexyl, heptyl, octyl, nonyl, decyl, the n isomers of these radicals, isopropyl, isobutyl, isopentyl, sec-butyl, tert-butyl, neopentyl, 3,3-dimethylbutyl. Both the main chain as well as the branches may furthermore contain heteroatoms as for instance N, O, S, Se or Si, or a carbon atom may be replaced by one of these heteroatoms. An aliphatic moiety may be substituted or unsubstituted with one or more functional groups. Substituents may be any functional group, as for example, but not limited to, amino, amido, carbonyl, carboxyl, hydroxyl, nitro, thio and sulfonyl.

In a more particular embodiment, the rhamnolipid(s) or rhamnolipid salts in said structure has the structure (II):

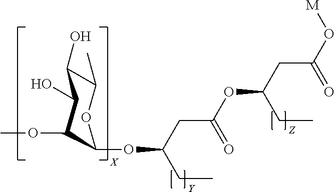

wherein x is 1 or 2, y is 4, 6 or 8, z is 4, 6, or 8, and M is H, or a metal, such as alkali metals Li, Na, or K, alkali earth metals Mg or Ca, or transition metals Mn, Fe, Cu, or Zn. In the cases of the alkali earth and transition metals, multiple rhamnolipid salt moieties may associate with each metal.

In some embodiments, the rhamnolipid component comprises a mixture of mono-rhamnolipids and di-rhamnolipids. The mono-rhamnolipids may be present in an amount of about 2% to about 60% by weight based on the total weight of rhamnolipids, alternatively about 2% to about 55% by weight, alternatively about 2% to about 50% by weight, alternatively about 3% to about 50% by weight, alternatively about 5% to about 48%, alternatively about 10% to about 48%, alternatively about 15% to about 48%, alternatively about 20% to about 48%, alternatively about 30% to about 48%, alternatively about 40% to about 48%, alternatively about 42% to about 48%, alternatively about 43% to about 48% alternatively about 10% to about 47%, alternatively about 15% to about 47%, alternatively about 20% to about 47%, alternatively about 30% to about 47%, alternatively about 40% to about 47%, alternatively about 43% to about 47%, alternatively about 5% to about 45% by weight, alternatively about 10% to about 45% by weight, alternatively about 25% to about 45% by weight, alternatively about 30% to about 45%, alternatively about 40% to about 45% by weight, alternatively about 43% to about 45% by weight based on the total weight of rhamnolipids.

The di-rhamnolipids may be present in an amount of about 40% to about 98% by weight based on the total weight of rhamnolipids, alternatively about 45% to about 98% by weight, alternatively about 50% to 98% by weight, alternatively about 50% to about 97% by weight, alternatively about 52% to about 90% by weight, alternatively about 52% to about 85%, alternatively about 52% to about 80%, alternatively about 52% to about 70%, alternatively about 52% to about 60%, alternatively about 52% to about 58%, alternatively about 52% to about 57%, alternatively about 53% to about 90% by weight, alternatively about 53% to about 85%, alternatively about 53% to about 80%, alternatively about 53% to about 70%, alternatively about 53% to about 60%, alternatively about 53% to about 57%, alternatively about 55% to about 95% by weight, alternatively about 55% to about 90% by weight, alternatively about 55% to about 75% by weight, alternatively about 55% to about 70% by weight, alternatively about 55% to about 60% by weight, alternatively about 55% to about 57% by weight, based on the total weight of rhamnolipids. The weight ratio of di-rhamnolipids:mono-rhamnolipids in the mixture can be from about 40:60 to about 98:2 alternatively about 50:50 to about 97:3. In some embodiments, the weight ratio of di-rhamnolipids:mono-rhamnolipids can range from 50:50 to 60:40, alternatively from 52:48 to 58:42, alternatively from 55:45 to 57:43, alternatively from 55:45 to 56.5:43.5. In other embodiments, the weight ratio of di-rhamnolipids to mono-rhamnolipids can be from 95:5 to about 98:2.

The mixture of rhamnolipids preferably comprises mono (where x=1) and di (where x=2) rhamnolipids where y and z are 6 and M is H or Na. The mono-rhamnolipid may be referred to as Rha-C10-C10, with a formula of C₂₆H₄₈O₉. The IUPAC Name is 3-[3-[(2R,3R,4R,5R,6S)-3,4,5-trihydroxy-6-methyloxan-2-yl]oxydecanoyloxy]decanoic acid. The di-rhamnolipid may be referred to as RhaRha-C10-C10, with a formula of C32H58013. The IUPAC name is 3-[3-[4, 5-dihydroxy-6-methyl-3-(3,4,5-trihydroxy-6-methyloxan-2-yl)oxyoxan-2-yl]oxydecanoyloxy]decanoic acid. Rha-C10-C10 may be present in the mixture in an amount of about 1% to about 5540% by weight, alternatively about 1% to about 50%, alternatively about 1% to about 45%, alternatively about 5% to about 45%, alternatively about 10% to about 45%, alternatively about 15% to about 45%, alternatively about 20% to about 45%, alternatively about 25% to about 45%, alternatively about 30% to about 45%, alternatively about 35% to about 45%, alternatively about 1% to about 40%, alternatively about 5% to about 40%, alternatively about 10% to about 40%, alternatively about 15% to about 40%, alternatively about 20% to about 40%, alternatively about 25% to about 40%, alternatively about 30% to about 40%, alternatively about 35% to about 40%, alternatively about 35% to about 37% by weight based on the total weight of rhamnolipids. RhaRha-C10-C10 may be present in the mixture in an amount of about 2530% to about 95%, alternatively about 30% to about 95%, alternatively about 30% to about 90%, alternatively about 30% to about 80%, alternatively about 30% to about 75%, alternatively about 30% to about 70%, alternatively about 30% to about 55%, alternatively about 30% to about 50%, alternatively about 30% to about 40%, alternatively about 35% to about 40%, alternatively about 36% to about 38% by weight based on the total weight of rhamnolipids.

In addition to Rha-C10-C10 and RhaRha-C10-C10, the mixture of rhamnolipids may comprise RhaRha-C10-C12 in an amount of about 1% to about 25% by weight, alternatively about 5% to about 15%, alternatively about 8% to about 12% by weight based on the total weight of rhamnolipids, and Rha-C10-C12 in an amount of about 0.2% to about 6% by weight, alternatively about 2% to about 5%, alternatively about 3.5% to about 5% by weight based on the total weight of rhamnolipids. The mixture of rhamnolipids may also comprise RhaRha-C10-C12:1 in an amount of about 0.2% to about 5% by weight, alternatively 1% to about 4% by weight, based on the total weight of rhamnolipids, an amount of RhaRha-C8-C10 in the range of about 0.2% to about 5% by weight, alternatively about 1% to about 4% by weight, based on the total weight of rhamnolipids, and an amount of Rha-C8-C10 in the range of about 0.2% to about 5% by weight, alternatively about 1% to about 4% by weight, based on the total weight of rhamnolipids.

The rhamnolipids may be produced from a rhamnolipid-producing microorganism that has the capacity to synthesize/produce rhamnolipids under suitable conditions. Such microorganisms include, but are not limited to, bacteria, particularly bacteria of the phyla Pseudomonadota, Actinobacteria, Fimicutes, and Proteobacteria. The rhamnolipids are naturally derived and therefore have a BCI of 100. In a particular embodiment, the rhamnolipid-producing microorganism for producing the rhamnolipids is Pseudomonas aeruginosa. Methods of culturing the rhamnolipid-producing bacteria and the production of rhamnolipids from fermentation are known in the art from, for example U.S. Pat. Nos. 11,142,782 and 10,144,943, incorporated herein by reference in their entirety. Methods of purifying the rhamnolipids are also known in the art from, for example, U.S. Pat. Nos. 9,884,883 and 10,829,507, incorporated herein by reference in their entirety.

The amount of rhamnolipid component in the hard surface cleaning compositions may be in the range of 0.10% to 0.55% active weight, based on the total weight of the composition. In some embodiments, the amount of rhamnolipids may be in the range of 0.38% to 0.44% by active weight.

The hard surface cleaning compositions also include at least one alkyl sarcosinate anionic surfactant, including sodium, potassium, magnesium, ammonium, monoethanolammonium, diethanolammonium, and triethanolammonium salts thereof. Preferably, the alkyl sarcosinate anionic surfactant is derived from a natural source, and has a BCI of at least 80, alternatively at least 90, alternatively at least 95, and preferably 100. Suitable alkyl sarcosinate surfactants for use in the compositions of the present technology are C8-C18 alkyl sarcosinates, such as sodium lauroyl sarcosinate. A specific example of a suitable alkyl sarcosinate surfactant is MAPROSYL® 30-B (sodium lauroyl sarcosinate), commercially available from Stepan Company, Northfield IL.

The amount of alkyl sarcosinate surfactant in the hard surface cleaning compositions can be in the range of 0.25% to less than 0.7% active weight, based on the total weight of the composition. Higher amounts of alkyl sarcosinate surfactant can result in unacceptable filming/streaking for the hard surface cleaner compositions. In some embodiments, the amount of alkyl sarcosinate surfactant may be 0.25% to 0.53% by active weight.

The cleaning compositions of the present technology include a builder or chelant as a buffering, softening, and emulsifying agent, which also neutralizes any metal ions present in the composition. Preferred builders for use herein are those that are derived from a natural source. Particularly preferred builders comprise gluconic acid, sodium gluconate, or a combination thereof, which are fermentation-based builders. Gluconic acid and sodium gluconate can have a BCI of at least 85, alternatively at least 90, alternatively at least 95. The amount of builder in the hard surface cleaning compositions should be in the range of 0.1% to less than 0.5%. Without a builder, the hard surface cleaner composition may have poor cleaning properties and/or unacceptable filming/streaking. An amount of builder of 0.5% or greater may result in poor cleaning performance. In some embodiments, the amount of the builder may be 0.1% to 0.3% by active weight.

The cleaning compositions of the present technology can be in liquid form, and comprise at least one carrier to bring the total percentage of the composition to 100%. As will be appreciated by at least those skilled in the art, a variety of carriers, vehicles, diluents, and the like are suitable for use in the practice of the present technology. Thus, it will also be appreciated that the terms “carrier”, “vehicle”, and “diluent” are to be considered non-exhaustive and interchangeable with respect to the present technology and in describing the various formulations, applications, uses, and compositions thereof.

Water is a suitable carrier, particularly for a Ready-to-Use formulation, and can be de-ionized water, hard water, soft water, distilled water, tap water or combinations thereof. Water can be used alone as the carrier, or in combination with other suitable carriers, such as for example, water-miscible solvents, such as alcohols or glycol ethers.

The hard surface cleaning compositions of the present technology can include optional ingredients as known in the art. Such other components or additives can include pH adjustment agents, hydrotropic or other solubilizing agents for obtaining and maintaining a clear single-phase concentrate or ready-to-use composition, electrolytes for enhancement of surfactant detergency, fragrances for different attractive smells, dyes for pleasing color, preservatives, and other functional ingredients.

The hard surface cleaning compositions of the present technology can be prepared, for example, as a ready-to-use product or dilutable concentrate product. Whether in a ready-to-use form or a dilutable concentrate, the end use concentration of the components are equivalent.

As defined above, a dilutable concentrate product is a product that requires dilution with a diluent (e.g., water) in a ratio of about, for example, 1:64, 1:32, 1:16 or 1:10 among others, before it can be applied to articles or surfaces to be cleaned. In some embodiments, dilutable cleaning compositions are preferred as a cost saving and money saving option, which reduces packaging and shipping cost. In some embodiments, the concentrate may be diluted to the working concentration on site and packaged as a ready to use liquid or spray. The dilutable concentrate can be a liquid or in the form of a powder. When the hard surface cleaner composition is in the form of a dilutable concentrate or powder, the amount of the at least one rhamnolipid in the composition is about 1% to about 36% by weight, based on the weight of the composition, the amount of the at least one alkyl sarcosinate surfactant is about 2.5% to less than 45% by weight, based on the weight of the composition, and the amount of the at least one builder is 1% to less than 32% by weight based on the weight of the composition.

The diluent for diluting the concentrate form of the composition can be any diluent system known in the art. Examples of suitable diluents include, but are not limited to, water, glycols (preferably propylene glycol), alcohols (e.g., isopropanol, ethanol, methanol), other polar solvents known in the art, and mixtures thereof. Water is a preferred diluent of the presently described technology, and can be de-ionized water, hard water, soft water, distilled water, tap water or combinations thereof.

The hard surface cleaning compositions of the present technology can have pH values in the range of from about 6 to about 8; alternatively, from about 6 to about 7. Solubility of the rhamnolipid component in water decreases as the pH is lowered from 7 to 6 as the rhamnolipid protonates. If the pH of the desired cleaning composition is lower than 7, it is preferable to prepare the composition by adding the anionic surfactant to the carrier first, and then adding the rhamnolipid component, to avoid precipitation of the rhamnolipid. The surfactant helps to dissolve the rhamnolipid to produce a phase stable, clear solution. Standard blending equipment is acceptable for preparing the cleaning compositions of the present technology.

Unexpectedly, the ability of the cleaning composition to provide a combination of good cleaning properties and low film/streak does not necessarily correlate with increased or decreased amounts of ingredients in the composition. Moreover, although varying the ratios and amounts of components typically provides linear small changes in performance, more (or less) of one or more of the components in the compositions of the present technology can result in unacceptable cleaning or unacceptable filming/streaking, or both, as demonstrated in the examples the follow. Thus, it is unpredictable what concentrations of the components would provide a combination of good cleaning ability and low filming/streaking outside of the ranges described herein. Also surprising is that substituting a different surfactant for the alkyl sarcosinate surfactant in a formulation having component amounts within the ranges described herein can result in a composition that does not provide effective cleaning properties, film/streak properties, or both. It is believed that it is the specific combination of the specific concentrations of the rhamnolipid component, the alkyl sarcosinate anionic surfactant, and the builder in the compositions of the present technology that results in cleaning compositions that provide an unexpected combination of effective cleaning and low filming/streaking.

The compositions and/or formulations of the present technology can be used for soil removing applications such as, but not limited to, cleaning bathroom and/or kitchen articles, appliances, surfaces and/or floors, glass/mirror surfaces, and other household surfaces. Institutional use in facilities, hotels, schools, etc. is also envisioned. The hard surface cleaning composition may be used to clean a surface by applying the composition to a soiled surface in an amount effective for removing the soil from the surface, and subsequently wiping the surface with a cloth, a wipe, a wiping device, or the like.

In some embodiments of the present application, the hard surface cleaning composition is envisioned to be used as a spray. The composition may be used as a spray in an RTU formulation, or a concentrate formulation can be used as a spray using, for example, a 1:8, 1:10, or a 1:32 dilution of the cleaning composition. Delivery devices can include a trigger spray, aerosol spray, pump spray, or other delivery device, such as a mop, cloth, brush, etc. In some embodiments, the composition may be used in a wipe impregnated with the composition. In other embodiments, the composition may be used, for example, in a wipe used with an applicator pad. In some embodiments, the composition of the present application is envisioned to be a concentrate that can be packaged, for example, in a packet or pod that can be added to water at an appropriate dilution ratio.

One skilled in the art will recognize that modifications may be made in the present technology without deviating from the spirit or scope of the invention. The invention is further illustrated by the following examples, which are not to be construed as limiting the invention in spirit or scope to the specific procedures or compositions described therein.

The following test methods are used to determine cleaning performance and film/streak properties of the compositions prepared in the Examples: Cleaning performance is conducted per ASTM D4488-95 A5, and film/streak performance is conducted per the method for determining film/streak disclosed in US Published Application No. 2021/0139815, incorporated herein by reference, where the results reported here are divided by a factor of 10.

Design-Expert Version 9.0.2.0 by Stat-Ease was used to model the cleaning test and film streak test results from each data set. The models of the cleaning and film streak tests were then used to generate an optimization plot using Design-Expert's Numerical Optimization function. The optimization includes a contour plot and a 3-D surface plot of “desirability” of component levels for the defined design space. The “desirability” is calculated by Design-Expert as a combination of the cleaning and film streak performance. These plots visually represent the complex relationships between the components and show the distinct peaks and valleys where desired performance is high and where it is low or zero. These peaks and valleys of desired performance show that it is the specific combination of the specific concentrations of the rhamnolipid component, the alkyl sarcosinate anionic surfactant, and the builder in the compositions of the present technology that results in cleaning compositions that provide an unexpected combination of effective cleaning and low filming/streaking.

### EXAMPLES

### Example 1: Hard Surface Cleaning Formulations

Table 1 describes hard surface cleaning formulations used to prepare ten hard surface cleaning compositions. The compositions were prepared by adding the rhamnolipid component as the last step, to a blend of water, anionic surfactant component, and builder component, then adjusting the pH to about 6. The anionic surfactant is MAPROSYL® 30-B, sodium lauroyl sarcosinate, available from Stepan Company, Northfield, Illinois, and the builder is sodium gluconate. The rhamnolipid component is a mixture of mono-rhamnolipids and di-rhamnolipids, in a weight ratio of di-rhamnolipids to mono-rhamnolipids in the range of 55:45 to 56.5:43.5. The mixture includes RhaRha-C10-C10 in an amount in the range of 36%-38% by weight, and Rha-C10-C10 in an amount in the range of 35%-37% by weight, based on the total weight of rhamnolipids in the mixture of rhamnolipids. All component amounts are in % active weight. Compositions 1A-3A are according to the present technology and have component amounts within the ranges described herein. Comparative compositions 1A-7A are not according to the present technology and have components that are missing and/or that are outside of the desired component ranges.

TABLE 1

| Formulation | Rhamnolipid | Surfactant | Builder | pH |
|---|---|---|---|---|
| Composition 1A | 0.5 | 0.3 | 0.2 | 6.07 |
| Composition 2A | 0.307 | 0.498 | 0.196 | 6.05 |
| Composition 3A | 0.102 | 0.511 | 0.387 | 6.03 |
| Comparative 1A | 0.5 | 0.5 | 0 | 6.05 |
| Comparative 2A | 0.5 | 0.5 | 0 | 6.05 |
| Comparative 3A | 0.1 | 0.7 | 0.2 | 6.05 |
| Comparative 4A | 0.1 | 0.7 | 0.2 | 6.06 |
| Comparative 5A | 0.2 | 0.3 | 0.5 | 6.03 |
| Comparative 6A | 0.2 | 0.3 | 0.5 | 6.01 |
| Comparative 7A | 0.3 | 0.7 | 0 | 6.06 |

### Example 2: Assessment of Cleaning Performance and Film/Streak

Each of the compositions from Example 1 was tested for cleaning performance and for filming/streaking using the test procedures described above. The cleaning performance results are reported as the percentage of soil removed after the first cleaning stroke and percentage of soil removed after the fifth cleaning stroke. Removal of at least 75% of soil after 7 cleaning strokes demonstrates good cleaning performance for this composition data set. Film/streak results compare the amount of streaking and filming for the composition compared to a base tile after application to a ceramic substrate and a polypropylene substrate. The film/streak testing for each formulation is run in triplicate on each substrate, and the triplicate run results are averaged and divided by a factor of 10. A value of 1.0 or less on ceramic and 3.0 or less on polypropylene is an indication of low filming/streaking for the composition. The test results are shown in Table 2.

TABLE 2

| | Cleaning | Cleaning | Cleaning | Film/ | Film/ |
|---|---|---|---|---|---|
| Formulation | % | % | % | Streak | Streak |
| pH 6 | Stroke 1 | Stroke 5 | Stroke 7 | Ceramic | PP |
| Composition 1A | 12.6 | 74.7 | 85.7 | 0.9 | 1.0 |
| Composition 2A | 10.3 | 61.1 | 77.9 | 0.9 | 2.0 |
| Composition 3A | 13.0 | 63.3 | 74.7 | 1.0 | 0.8 |
| Comparative 1A | 16.2 | 77.6 | 86.3 | 1.2 | 0.7 |
| Comparative 2A | 14.2 | 66.3 | 79.2 | 3.0 | 0.7 |
| Comparative 3A | 18.8 | 78.2 | 85.8 | 3.4 | 1.2 |
| Comparative 4A | 14.4 | 68.5 | 78.5 | 3.6 | 1.1 |
| Comparative 5A | 8.9 | 42.3 | 57.5 | 0.8 | 0.7 |
| Comparative 6A | 8.6 | 41.9 | 58.8 | 0.7 | 1.1 |
| Comparative 7A | 10.6 | 58.5 | 67.9 | 3.6 | 1.0 |

The results in Table 2 show that the three compositions in accordance with the present technology have both good cleaning performance, as shown by stroke 7 soil removal of about 75% or more, and low film/streak, as shown by values of 1.0 or less for ceramic and 3.0 or less on polypropylene surfaces. Composition 3 had 74.7% soil removal by stroke 7 and is still considered an example of the present invention because the result is within the accepted error of the method. By contrast, the comparative examples had either poor cleaning, poor film/streak properties on the ceramic surface, or both. The results show that poor film/streak properties on ceramic occurred in compositions that did not include a builder, and in compositions that had an amount of alkyl sarcosinate surfactant of 0.7%. The results also show that the comparative examples with a builder amount of 0.5% had a clear drop off in cleaning performance.

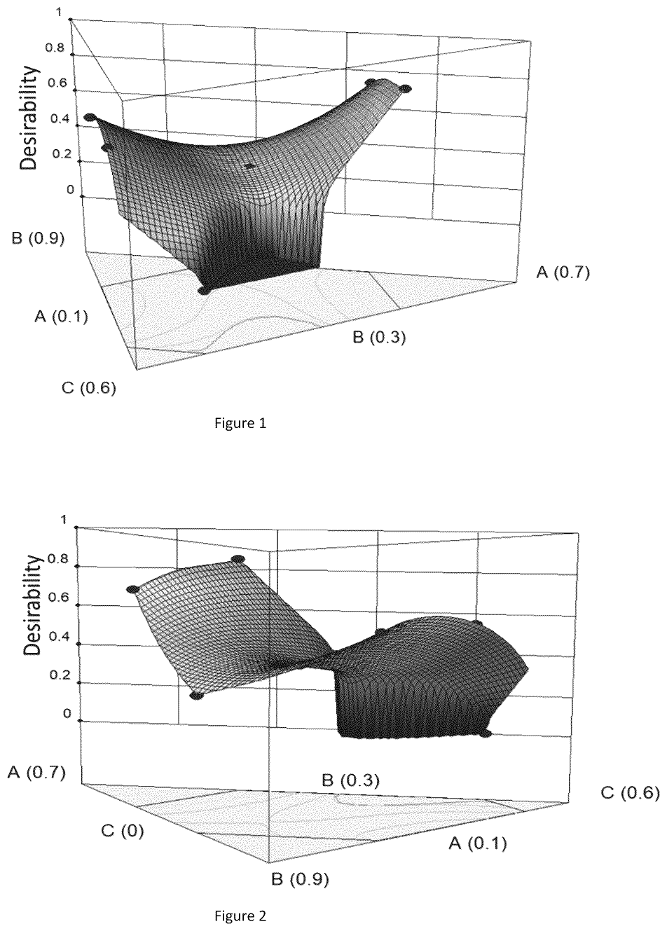

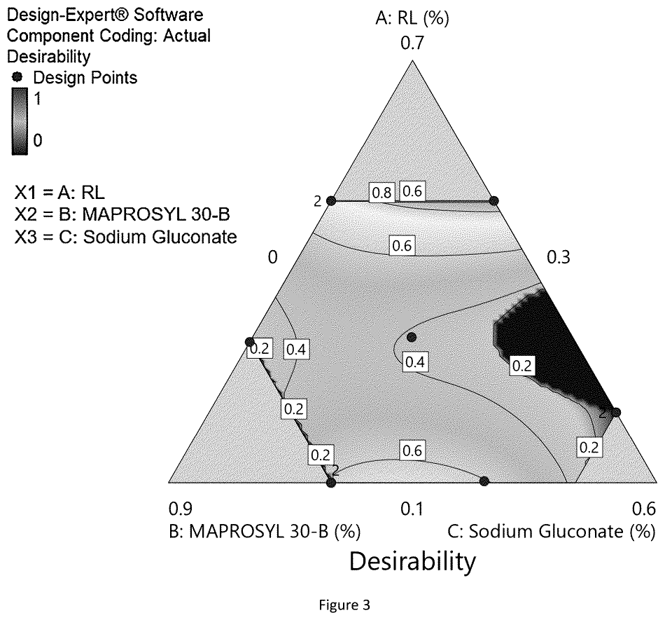

The Stat-Ease Design-Expert optimization 3-D surface plot for the cleaning formulations in Table 1, shown in FIGS. 1 and 2, has a saddle shape and shows areas of high desirability with a downward slope to areas of lower desirability that then increase in slope along the saddle shaped region to another area of higher desirability. This 3-D surface plot also has a region with a very steep slope down to a distinct area of very low or zero desirability of formulations, best shown in FIG. 2. These areas of very low desirability appear as a floor at the base of the optimization 3-D surface plot. The areas of high to medium to low desirability can also be seen in the optimization contour plot found in FIG. 3 by the numerical values that show the contour lines in the plot. These regions of high to very low desirability show that it is the specific combination of the specific concentrations of the rhamnolipid component, the anionic surfactant, and the builder in the compositions of the present technology that results in cleaning compositions that provide an unexpected combination of effective cleaning and low filming/streaking.

### Example 3: Hard Surface Cleaning Formulations-Alternative Surfactants (Comparative)

Table 3 describes hard surface cleaning formulations in which the alkyl sarcosinate surfactant is replaced by an alternative anionic or amphoteric surfactant. The compositions were prepared by adding the rhamnolipid component as the last step, to a blend of water, anionic or amphoteric surfactant component, and builder component, then adjusting the pH to about 6. The alternative surfactants are sodium lauryl sulfate anionic surfactant (Stepanol® 1B) WA-Extra) (Comparative and cocoamidopropyl hydroxysultaine amphoteric surfactant (AMPHOSOL® CS-50) (Comparative 2B), all commercially available from Stepan Company, Northfield, IL. The builder is sodium gluconate, and the rhamnolipid component is a mixture of mono-rhamnolipids and di-rhamnolipids, in a weight ratio of di-rhamnolipids to mono-rhamnolipids in the range of 55:45 to 56.5:43.5. The mixture includes RhaRha-C10-C10 in an amount in the range of 36%-38% by weight, and Rha-C10-C10 in an amount in the range of 35%-37% by weight, based on the total weight of the rhamnolipids in the mixture of rhamnolipids. All component amounts are in % active weight. Comparative formulations 1B and 2B, and composition 2A contain the same amounts of rhamnolipid, surfactant, and builder components.

TABLE 3

| Formulation | Rhamnolipid | Surfactant | Builder | pH |
|---|---|---|---|---|
| Comparative 1B | 0.307 | 0.498 | 0.196 | 6.05 |
| (Alkyl Sulfate) | | | | |
| Comparative 2B | 0.307 | 0.498 | 0.196 | 6.02 |
| (CS-50) | | | | |
| Composition 2A | 0.307 | 0.498 | 0.196 | 6.05 |
| (Sarcosinate) | | | | |

### Example 4: Assessment of Cleaning Performance and Film/Streak

Each of the compositions from Example 3 was tested for cleaning performance and for filming/streaking using the same test procedures described above. The test results are shown in Table 4.

TABLE 4

| | Cleaning | Cleaning | Cleaning | Film/ | Film/ |
|---|---|---|---|---|---|
| Formulation | % | % | % | Streak | Streak |
| pH 6 | Stroke 1 | Stroke 5 | Stroke 7 | Ceramic | PP |
| Comparative 1B | 34.6 | 79.0 | N/A | 3.2 | 1.1 |
| Comparative 2B | 6.6 | 54.8 | 74.0 | 0.4 | 0.9 |
| Composition 2A | 10.3 | 61.1 | 77.9 | 0.9 | 2.0 |

The results in Table 4 show that the compositions of the present technology, using alkyl sarcosinate as the co-surfactant, have both good cleaning performance, as shown by stroke 7 soil removal of about 75% or more, and low film/streak, as shown by values of 1.0 or less for ceramic and 3.0 or less polypropylene surfaces. By contrast, neither of the comparative formulations showed a combination of good cleaning and low film/streak. Comparative 1B showed good cleaning but unacceptable film/streak performance, while Comparative 2B showed acceptable film/streak but poor cleaning. The results show that substituting other surfactants for the alkyl sarcosinate surfactant may not provide the same combination of properties, and further demonstrate that it is the specific combination of the specific concentrations of the rhamnolipid component, the alkyl sarcosinate anionic surfactant, and the builder in the compositions of the present technology that results in cleaning compositions that provide an unexpected combination of effective cleaning and low filming/streaking.

The embodiments and examples described here are illustrative, and do not limit the presently described technology in any way. The scope of the present technology described in this specification is the full scope defined or implied by the claims. Additionally, any references noted in the detailed description section of the instant application are hereby incorporated by reference in their entireties, unless otherwise noted.

The present technology is now described in such full, clear and concise terms as to enable a person skilled in the art to which it pertains, to practice the same. It is to be understood that the foregoing describes preferred embodiments of the present technology and that modifications may be made therein without departing from the spirit or scope of the present technology as set forth in the appended claims. Further the examples are provided to not be exhaustive but illustrative of several embodiments that fall within the scope of the claims.

What is claimed is:

1. A hard surface cleaner composition comprising:
(1) at least one rhamnolipid in an amount of 0.10% to 0.55% active weight based on the total weight of the composition;
(2) at least one alkyl sarcosinate anionic surfactant in an amount of 0.25% to less than 0.7% active weight based on the total weight of the composition;
(3) at least one builder in an amount of 0.1% to less than 0.50% active weight based on the total weight of the composition; and
(4) at least one carrier in an amount to total 100% by weight of the composition,
wherein the at least one builder comprises gluconic acid, sodium gluconate, or a combination thereof.

2. The hard surface cleaner composition of claim 1, wherein the at least one rhamnolipid is a mixture of rhamnolipids.

3. The hard surface cleaner composition of claim 2, wherein the mixture of rhamnolipids comprises mono-rhamnolipids and di-rhamnolipids.

4. The hard surface cleaner composition of claim 3, wherein the mixture of rhamnolipids has a weight ratio of di-rhamnolipids to mono-rhamnolipids of 50:50 to 98:2.

5. The hard surface cleaner composition of claim 1, wherein the alkyl sarcosinate anionic surfactant comprises sodium lauryl sarcosinate.

6. The hard surface cleaner composition of claim 1, wherein the amount of alkyl sarcosinate anionic surfactant is 0.25% to 0.53% by active weight.

7. The hard surface cleaner composition of claim 1, wherein the amount of rhamnolipid is 0.38% to 0.44% by active weight.

8. The hard surface cleaner composition of claim 1, wherein the amount of builder is 0.1% to 0.3% by active weight.

9. The hard surface cleaner composition of claim 1, wherein the carrier comprises water.

10. The hard surface cleaner composition of claim 1, wherein the composition has a pH of 6.0 to 8.0.

11. A method of cleaning a hard surface comprising:
contacting at least one soiled surface with a composition according to claim 1, and
removing the composition and soil from the surface.

12. The method of claim 11, wherein the composition is applied to the soiled surface by spraying the composition on the surface.

13. The method of claim 11, wherein the composition is applied to the soiled surface by wiping the surface with a wipe that is impregnated with the composition.

14. A dilutable hard surface cleaner composition comprising:
(1) at least one rhamnolipid in an amount of 1.0% to 36% active weight based on the total weight of the composition;
(2) at least one alkyl sarcosinate anionic surfactant in an amount of 2.5% to less than 45% active weight based on the total weight of the composition;
(3) at least one builder in an amount of 1.0% to less than 32% active weight based on the total weight of the composition; and
(4) at least one carrier in an amount to total 100% by weight of the composition,
wherein the at least one builder comprises gluconic acid, sodium gluconate, or a combination thereof.

15. The dilutable hard surface cleaner composition of claim 14, wherein the dilutable hard surface cleaner composition is diluted at a ratio of 1:10, 1:16, 1:32 or 1:64 in a diluent.

16. The dilutable hard surface cleaner composition of claim 14, wherein the diluent is water.

17. The dilutable hard surface cleaner of claim 14, wherein the dilutable hard surface cleaner composition is a liquid concentrate or a powder.

18. The dilutable hard surface cleaner of claim 14, wherein the at least one rhamnolipid comprises a mixture of mono-rhamnolipids and di-rhamnolipids in a weight ratio of di-rhamnolipids to mono-rhamnolipids of 50:50 to 98:2.
